(19) United States
(12) Patent Application Publication
(10) Pub. No.: US 2024/0398317 A1
(43) Pub. Date: Dec. 5, 2024

(54) METHOD AND SYSTEM TO DECODE SPEECH PRODUCTION FROM NON-FRONTAL, NON-POST-CENTRAL BRAIN CORTICES

(71) Applicant: Northwestern University, Evanston, IL (US)

(72) Inventors: Marc W. Slutzky, Northbrook, IL (US); Prashanth Ravi Prakash, Chicago, IL (US)

(21) Appl. No.: 18/731,859

(22) Filed: Jun. 3, 2024

Related U.S. Application Data
(60) Provisional application No. 63/471,056, filed on Jun. 5, 2023.

(51) Int. Cl. A61B 5/369; A61B 5/291

(52) U.S. Cl. CPC A61B 5/369; A61B 5/291

(57) ABSTRACT

A system to decode communication signals includes a memory configured to store brain signals, where the brain signals originate from non-frontal, non-post-central cortices of a brain of a person. The system also includes a processor operatively coupled to the memory. The processor is configured to perform signal processing on the brain signals to identify one or more brain signal features. The processor is also configured to determine, based on the one or more identified brain signal features, whether the person intends to speak. Responsive to a determination that the person intends to speak, the processor identifies phonemes corresponding to the brain signals.

### CROSS-REFERENCE TO RELATED APPLICATION

The present application claims the priority benefit of U.S. Provisional Patent App. No. 63/471,056 filed on Jun. 5, 2023, the entire disclosure of which is incorporated herein by reference.

### REFERENCE TO GOVERNMENT RIGHTS

This invention was made with government support under CA221747 awarded by the National Institutes of Health. The government has certain rights in the invention.

### BACKGROUND

A brain-computer interface (BCI), also called a brain machine interface (BMI), refers to a computing system that utilizes a direct communication pathway between a computer and a human brain. Signals generated by the brain are received at the computer via the interface. The computer processes the received signals and controls an external device based on the processed signals. The external device can be a robotic limb that is used to restore movement to an individual with paralysis or another ailment that prevents proper limb function. The external device can also be a speech processing system that converts the processed signals into text or speech to assist individuals that have difficulty speaking.

### SUMMARY

An illustrative system to decode communication signals includes a memory configured to store brain signals, where the brain signals originate from non-frontal, non-post-central cortices of a brain of a person. The system also includes a processor operatively coupled to the memory. The processor is configured to perform signal processing on the brain signals to identify one or more brain signal features. The processor is also configured to determine, based on the one or more identified brain signal features, whether the person intends to speak. Responsive to a determination that the person intends to speak, the processor identifies phonemes of the intended spoken words using the brain signal features.

The system can also include an array of electrodes implanted on or in the person's brain such that the brain signals are received from the array of electrodes. In one embodiment, the processor is further configured to combine the identified phonemes to generate words corresponding to the brain signals. In another embodiment, responsive to a determination that the person does not intend to speak at a given time point, the processor does not decode thoughts of the person that occur at the given time point. In another embodiment, the brain signals originate from one or both of the temporal lobe of the person and the parietal lobe of the person. The brain signals can be high gamma band signals and/or action potentials in the form of spikes. In another embodiment, the brain signals are low-frequency signals in a range between 0 Hertz and 30 Hertz. In another embodiment, a frontal lobe of the patient is damaged such that the frontal lobe does not generate brain signals that result in speech.

An illustrative method of processing speech includes storing, in a memory of a computing device, brain signals that originate from non-frontal, non-post-central cortices of brains of individuals. The method also includes performing, by a processor of the computing device, signal processing on the brain signals. The method also includes generating, by the processor, binned features based on the signal processing of the brain signals. The method also includes performing, by the processor, feature selection on the binned features to identify a spatial pattern associated with speech. The method further includes generating, by the processor, based on the spatial pattern, a first decoder to predict intent to speak of an individual.

In one embodiment, the method further includes generating, based on the feature selection, a second decoder of phoneme classes corresponding to the brain signals. The method can also include comprising receiving an additional brain signal and using the first decoder to determine whether the additional brain signal represents an intent to speak. The additional brain signal is from a patient, and the frontal lobe of the patient is damaged such that the frontal lobe does not generate brain signals that result in speech. The method can also include, responsive to a determination that the additional brain signal represents the intent to speak, using the second decoder to identify one or more phonemes associated with the additional brain signal.

The method can also include, responsive to a determination that the additional brain signal does not represent the intent to speak, taking no action to avoid reading thoughts. In one embodiment, the brain signals comprise action potentials or spike band signals (e.g., 300-1000 Hertz (Hz)). In another embodiment, the brain signals comprise high gamma band (e.g., 70-300 Hz) signals. In an illustrative embodiment, the brain signals are received from arrays of electrodes attached to brains of a plurality of individuals. The brain signals originate from one or both of the temporal lobe and the parietal lobe of the plurality of individuals. In another embodiment, the binned features have a duration between 50 milliseconds and 100 milliseconds.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts classification accuracy of speech versus silence using the proposed system in accordance with an illustrative embodiment.

FIG. 2 depicts the results of decoding between speech and silence for individual patients in accordance with an illustrative embodiment.

FIG. 3A depicts results of decoding between plosive phonemes and nasal phonemes in accordance with an illustrative embodiment.

FIG. 3B depicts results of decoding between nasal phonemes and liquid phonemes in accordance with an illustrative embodiment.

FIG. 3C depicts results of decoding between fricative phonemes and nasal phonemes in accordance with an illustrative embodiment.

FIG. 3D depicts results of decoding between nasal phonemes and glide phonemes in accordance with an illustrative embodiment.

FIG. 4 is a flow chart depicting operations performed by a computing system to decode speech production from non-frontal, non-post-central brain cortices in accordance with an illustrative embodiment.

FIG. 5 is a block diagram of a computing system to implement a system to decode intent to speak signals and speech signals from non-frontal, non-post-central brain cortices in accordance with an illustrative embodiment.

### DETAILED DESCRIPTION

Brain-computer interfaces (BCIs) are an emerging technology that can potentially be used to improve the lives of individuals with physical disabilities, speech impediments, paralysis, and other injuries. Traditional BCIs aiming to decode speech production to restore communication have largely recorded signals from the speech sensorimotor cortices, including ventral pre-central and postcentral gyri and inferior frontal gyrus. The temporal and the parietal lobes are also important areas of interest for speech and language perception, but thus far there is a lack of evidence of a speech production signal in these areas of the brain. If a system can be designed to decode speech production from these areas, such a system could potentially be used to restore communication to people with communication disorders, including expressive aphasia, in which the frontal lobe is damaged. Described herein is a system that utilizes speech production signals that are based on electrocorticographic (ECoG) signals recorded from the temporal and parietal cortices of individuals. Also described herein are methods and systems for processing such signals and generating speech/text based on the processed signals.

To demonstrate existence of speech signals originating from the temporal and/or parietal cortices, a study was conducted in which ECoG electrode arrays were placed on the temporal and/or parietal cortices in participants undergoing resection of epileptic foci or brain tumors. In participants with epilepsy, standard arrays (10-millimeter (mm) interelectrode spacing) were placed according to clinical necessity. In participants with tumors, high-density ECoG arrays (8×8 array with 4-mm interelectrode spacing) were placed on the temporal and/or parietal lobes intraoperatively. Participants were presented with single words on a screen in random order. They were instructed to read each word silently, hold it in memory while viewing a blank screen, and then cued visually to say it out loud. This enabled the inventors to disentangle the ECoG signatures of speech production from those of reading or comprehension.

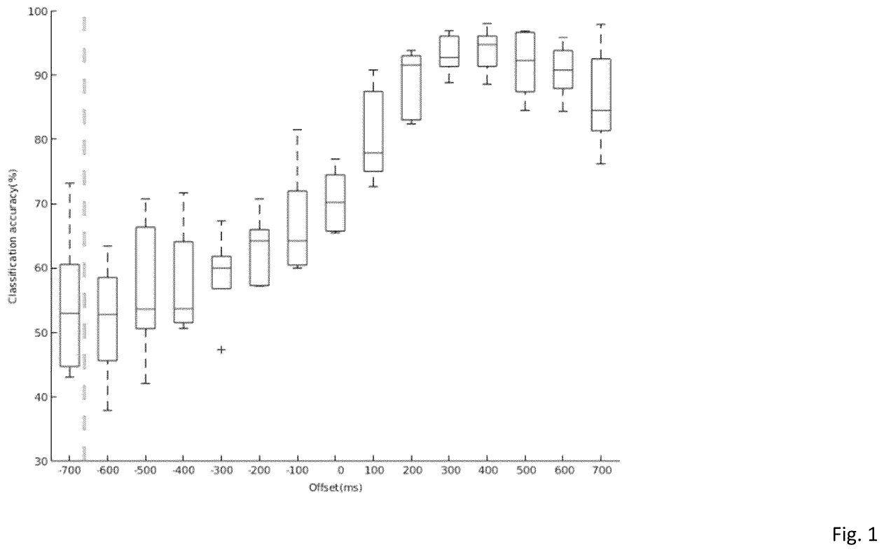

Speech intent decoding was also performed. Specifically, ECoG high-gamma (HG) band [70-300 Hertz (Hz)] power in 100-millisecond (ms) non-overlapping windows was used as a feature for decoding speech intent. Each window was labeled according to the respective behavioral state: speech or silence (resting state). FIG. 1 depicts classification accuracy of speech versus silence using the proposed system in accordance with an illustrative embodiment. Each box in FIG. 1 represents the median (horizontal line) and interquartile range of accuracy of decoding between speech and silent periods over the five participants. To avoid bias due to imbalanced classes and only include causal information, for every spoken word the inventors included either a speech window at the voice onset of that word or a silence window from 1.5-1.6 seconds(s) after the voice offset of the previous word. A radial basis function support vector machine was trained to classify the behavioral state of each window using a history of 4 windows. The offset between HG power and speech/silence window was varied from −1.5 s to 0.7 s, where negative values indicate HG leading behavior. The inventors also compared the results to chance accuracy by empirically shuffling the labels of the windows and building decoders with the shuffled data, using 100 shuffles.

Using this technique, speech vs. silence was decoded using only causal information (i.e., using windows up to the last HG window before speech/silence window onset) with accuracies ranging from 67.2%-80% over participants (p<0.03 in all participants, t-test, compared to shuffled labels). To further investigate evidence for a speech intent signal, demixed principal components analysis was used on the HG power from these cortices to reduce the dimensionality of the data set. The principal components were computed using HG data from −0.5 s to the onset of speech. A separation between the speech and silence behavioral states was observed in a lower dimensional space using the first 3 most significant demixed principal components. In alternative implementations, a different number of most significant demixed principal components can be used.

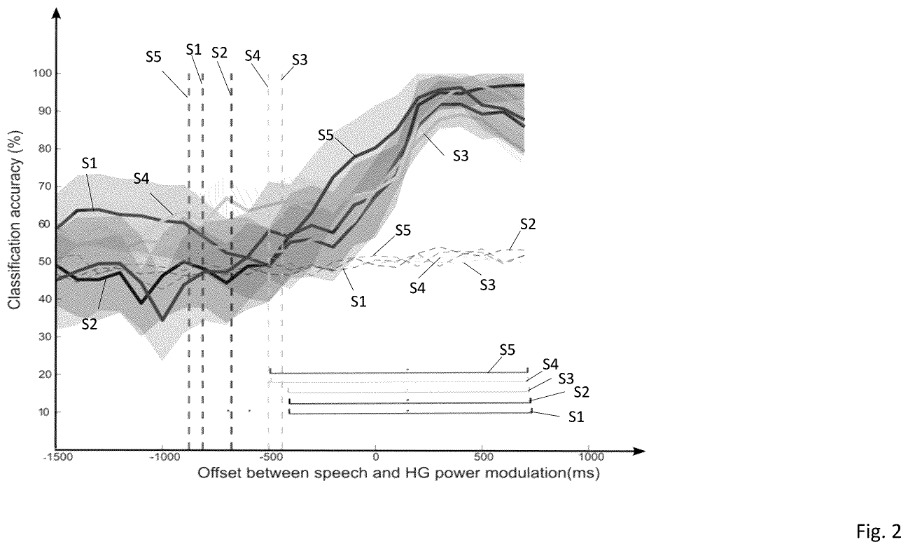

FIG. 2 depicts the results of decoding between speech and silence for individual patients in accordance with an illustrative embodiment. In FIG. 2, each solid line shows the decoding accuracy at each time point for a given patient. The generally horizontal dotted lines show chance performance, the vertical dashed lines show mean target word presentation times, and the shaded regions show the standard deviation of decoding performance. Additionally, brackets with asterisks at the bottom of the figure show time bins with significantly better performance than chance (measured at p<0.01 level using t-tests).

Phoneme decoding was also performed. ECoG high gamma band power in 100-ms non-overlapping windows were used as features. For every spoken word the phoneme in a window at the voice onset of that word was decoded. To avoid signals related to the patient hearing themselves speak, only the initial phoneme of the word being uttered was decoded. Based on manner of articulation, each initial phoneme was labeled with one of 5 phoneme classes-plosives, fricatives, nasals, glides, or liquids. For instance, if the word prompted was ‘pear,’ the corresponding label assigned was plosive because the initial /p/ is a plosive phoneme. The offset between HG power and voice onset was varied from −1.5 s to 0.7 s, and multiple decoders were built for each offset value. To simplify the analysis, pairwise decoders were built that decoded between all 10 pairs of phoneme classes. For every pair of phoneme classes, to avoid bias due to imbalanced class sizes, the inventors subsampled the phoneme class with greater number of samples to have the same number of samples as its pair. A 5-fold cross-validated, radial basis function support vector machine decoder was used for this process.

Using this method, the results indicate an ability to differentiate between plosives vs nasals, fricatives vs nasals, nasals vs liquids and nasal vs glides with accuracies significantly better than chance prior to the onset of speech. Decoding accuracies ranged from 72.2%-77.5% (p<0.003, t-test) using data from 500 to 100 ms before voice onset and from 81.3-85% using data from 400 to 0 ms before voice onset (p<0.002). This provides evidence of information about the manner of articulation in the temporal and parietal lobes preceding speech production.

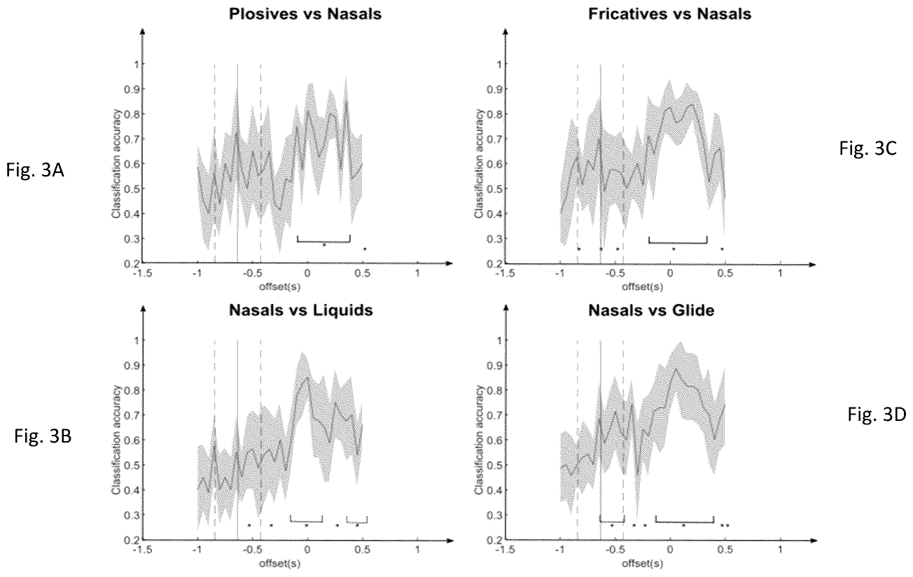

FIG. 3 depicts the results of decoding phoneme classes by manner of articulation. FIG. 3A depicts results of decoding between plosive phonemes and nasal phonemes in accordance with an illustrative embodiment. FIG. 3B depicts results of decoding between nasal phonemes and liquid phonemes in accordance with an illustrative embodiment. FIG. 3C depicts results of decoding between fricative phonemes and nasal phonemes in accordance with an illustrative embodiment. FIG. 3D depicts results of decoding between nasal phonemes and glide phonemes in accordance with an illustrative embodiment. As shown, decoding performance became better than chance starting at 150-200 ms prior to speech onset (time 0), suggesting that there is information about manner of articulation for production in non-frontal, non-post-central gyrus areas. Offsets marked with asterisks denote times at which decoding performance was significantly better than chance (p<0.004, t-test).

These results suggest that there is a speech production signal encoded within the temporal and parietal lobes (i.e., non-frontal and non-post-central gyrus brain cortices). This signal appears at least 200-300 ms prior to the onset of intended speech. The existence of such activity in the temporal lobe has been theorized in some linguistic speech production models, but there has been limited evidence for this activity to date. In addition, there appears to be causal information about produced phonemes, specifically the manner of their articulation, in the temporal and parietal lobes as well. These results are highly relevant to previously understudied cortical areas for spoken language production. Additionally, these results can be used advance the development of speech BCIs for people with communication disorders including language disorders (aphasia) as well as motor speech disorders (locked-in syndrome).

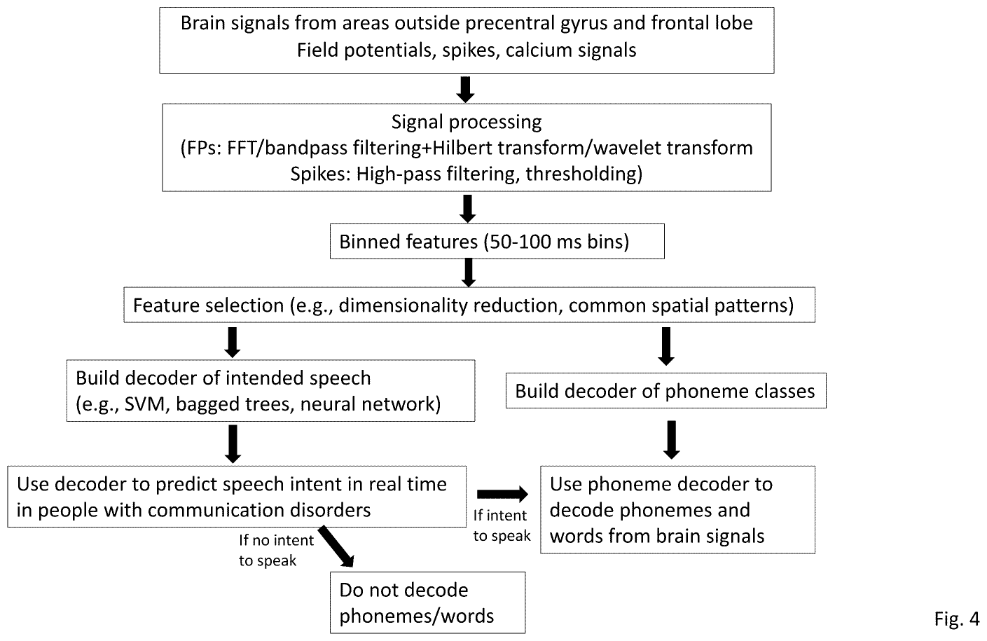

FIG. 4 is a flow chart depicting operations performed by a computing system to decode speech production from non-frontal, non-post-central brain cortices in accordance with an illustrative embodiment. In alternative embodiments, fewer, additional, and/or different operations may be performed. Additionally, the use of a flow diagram is not meant to be limiting with respect to the order of operations performed. As discussed in more detail below, the operations of the flow diagram can be performed by a computing system. In an illustrative embodiment, brain signals are received from areas outside of the frontal lobe of the brain (i.e., from non-frontal brain cortices). The signals can be received by one or more electrodes that are positioned on or proximate to the brain of a patient. Alternatively, the signals can be received through any other technique, such as image analysis of captured images of the brain. The received signals can be in the form of electrical field potentials, magnetic signals, spikes, calcium signals, etc.

Once received at the computing system, the brain signals are processed. For example, electrical field potential signals can be processed using a fast Fourier transform (FFT), bandpass filtering and a Hilbert transform, a wavelet transform, etc. Spikes can be processed using high-pass filtering, thresholding, etc. The computing system generates binned features in response to the processed brain signals. In one embodiment, the binned features can include bins that range in time from 50-100 ms. Alternatively, a different time value can be used, such as 10 ms, 20 ms, 30 ms, 120 ms, etc.

Feature selection is performed on the binned signal to perform dimensionality reduction, to identify common spatial patterns, etc. The feature selection process and the resulting generated data are used to help build both a decoder of intended speech and a decoder of phoneme classes. The decoder of intended speech can be built using a support vector machine (SVM), bagged trees, a neural network, or any other technique/algorithm. The decoder of intended speech is used to predict speech intent (i.e., intent to speak or intent to remain silent) based on subsequent received and processed brain signals. If the decoder of intended speech predicts no intent to speak (or intent to remain silent), the system does not decode any words or phonemes based on the brain signals. If the decoder of intended speech predicts an intent to speak, the phoneme or word decoder is used to decode phonemes and words from the processed brain signals.

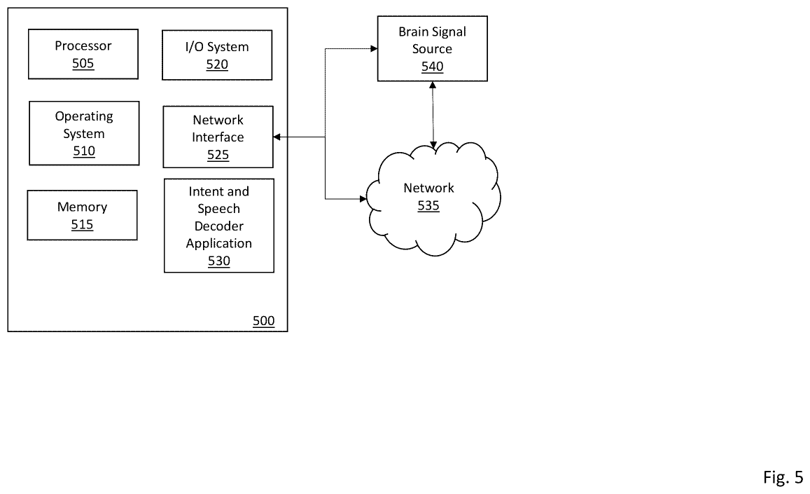

In an illustrative embodiment, any of the operations described herein can be performed by a computing system that includes a processor, a memory, a user interface, transceiver, etc. Any of the operations described herein can be stored in the memory as computer-readable instructions. Upon execution of these computer-readable instructions by the processor, the computing system performs the operations described herein. FIG. 5 is a block diagram of a computing system 500 to implement a system to decode intent to speak signals and speech signals from non-frontal, non-post-central brain cortices in accordance with an illustrative embodiment.

In one embodiment, the computing system 500 is in communication with a network 535 and a brain signal source 540. The computing system 500 can communicate directly with the brain signal source 540 or indirectly through the network 535. In an illustrative embodiment, the brain signal source 540 can be an electrode array that is positioned on, within, or proximate to the brain of a patient and used to collect electrical signals generated by the brain. In another illustrative embodiment, the electrode array is positioned to receive brain signals that originate from the non-frontal, non-post-central cortices of the brain. In an alternative embodiment, the brain signal source 540 can be an imaging system (e.g., MRI, ultrasound, optical imaging, magnetic imaging, etc.) that generates brain images from which brain signals can be extracted.

The computing system 500 includes a processor 505, an operating system 510, a memory 515, an input/output (I/O) system 520, a network interface 525, and an intent and speech decoder application 530. In alternative embodiments, the computing system 500 may include fewer, additional, and/or different components. The components of the computing system 500 communicate with one another via one or more buses or any other interconnect system. The computing system 500 can be any type of networked computing device. For example, the computing system 500 can be a smartphone, a tablet, a laptop computer, a dedicated device specific to the decoding applications, etc.

The processor 505 can be in electrical communication with and used to control any of the system components described herein. The processor 505 can be any type of computer processor known in the art, and can include a plurality of processors and/or a plurality of processing cores. The processor 505 can include a controller, a microcontroller, an audio processor, a graphics processing unit, a hardware accelerator, a digital signal processor, etc. Additionally, the processor 505 may be implemented as a complex instruction set computer processor, a reduced instruction set computer processor, an x86 instruction set computer processor, etc. The processor 505 is used to run the operating system 510, which can be any type of operating system.

The operating system 510 is stored in the memory 515, which is also used to store programs, user data, network and communications data, peripheral component data, brain signal data, the intent and speech decoder application 530, and other operating instructions. The memory 515 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 520 is the framework which enables users and peripheral devices to interact with the computing system 500. The I/O system 520 can include one or more displays (e.g., light-emitting diode display, liquid crystal display, touch screen display, etc.), a speaker, a microphone, etc. that allow the user to interact with and control the computing system 500. The I/O system 520 also includes circuitry and a bus structure to interface with peripheral computing devices such as power sources, USB devices, data acquisition cards, peripheral component interconnect express (PCIe) devices, serial advanced technology attachment (SATA) devices, high definition multimedia interface (HDMI) devices, proprietary connection devices, etc.

The network interface 525 includes transceiver circuitry (e.g., a transmitter and a receiver) that allows the computing system to transmit and receive data to/from other devices such as the brain signal source 540, other remote computing systems, servers, websites, etc. The data received from the camera 540 can include a plurality of captured images, image metadata, etc. The network interface 525 enables communication through the network 535, which can be one or more communication networks. The network 535 can include a cable network, a fiber network, a cellular network, a wi-fi network, a landline telephone network, a microwave network, a satellite network, etc. The network interface 525 also includes circuitry to allow device-to-device communication such as Bluetooth® communication.

The intent and speech decoder application 530 can include software and algorithms in the form of computer-readable instructions which, upon execution by the processor 505, performs any of the various operations described herein such as receiving brain signals, categorizing the brain signals, processing the brain signals using various filters and/or transforms, generating binned features, performing feature selection of the processed brain signal data, generating a decoder to determine speech intent based on the processed brain signals, generating a decoder to determine phonemes or words based on the processed brain signals, using the speech intent decoder to determine whether subsequently received brain signals represent an intent to speak or intent to remain silent, using the phoneme decoder to identify phonemes and words from the processed brain signals, etc. The intent and speech decoder application 530 can utilize the processor 505 and/or the memory 515 as discussed above. In an alternative implementation, the intent and speech decoder application 530 can be remote or independent from the computing system 500, but in communication therewith.

The methods and systems described herein can be useful in providing a method to communicate to people with aphasia using a BCI. In such patients, it is critical to be able to decode whether someone is trying to speak, or just thinking, because it is undesirable to decode the patient's thoughts. Thus, decoding intent to speak is a critical component of a speech BCI for such patients. In addition, the ability to decode speech-related information outside the frontal lobe is important in people with aphasia from stroke, traumatic brain injury, or degenerative conditions in which the frontal lobe is often damaged. As a result, existing techniques decoding from frontal areas are ineffective and much less likely to provide accurate results. The methods and systems described herein can also be useful in providing a method to communicate to people with other communication disorders, such as locked-in syndrome from brainstem stroke or motor neuron diseases.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system to decode communication signals, the system comprising:
a memory configured to store brain signals, wherein the brain signals originate from non-frontal, non-post-central cortices of a brain of a person; and
a processor operatively coupled to the memory, wherein the processor is configured to:
perform signal processing on the brain signals to identify one or more brain signal features;
determine, based on the one or more identified brain signal features, whether the person intends to speak; and
responsive to a determination that the person intends to speak, identify phonemes of the intended spoken words using the brain signal features.

2. The system of claim 1, further comprising an array of electrodes implanted on or in the person's brain, wherein the brain signals are received from the array of electrodes.

3. The system of claim 1, wherein the processor is further configured to combine the identified phonemes to generate words corresponding to the brain signals.

4. The system of claim 1, wherein, responsive to a determination that the person does not intend to speak at a given time point, the processor does not decode thoughts of the person that occur at the given time point.

5. The system of claim 1, wherein the brain signals originate from one or both of the temporal lobe of the person and the parietal lobe of the person.

6. The system of claim 1, wherein the brain signals comprise high gamma band signals.

7. The system of claim 1, wherein the brain signals comprise action potentials or spike band signals.

8. The system of claim 1, wherein the brain signals comprise low-frequency signals in a range between 0 Hertz and 30 Hertz.

9. The system of claim 1, wherein a frontal lobe of the patient is damaged such that the frontal lobe does not generate brain signals that result in speech.

10. A method of processing speech, the method comprising:
storing, in a memory of a computing device, brain signals that originate from non-frontal, non-post-central cortices of brains of individuals;
performing, by a processor of the computing device, signal processing on the brain signals;
generating, by the processor, binned features based on the signal processing of the brain signals;
performing, by the processor, feature selection on the binned features to identify a spatial pattern associated with speech; and
generating, by the processor, based on the spatial pattern, a first decoder to predict intent to speak of an individual.

11. The method of claim 10, further comprising generating, based on the feature selection, a second decoder of phoneme classes corresponding to the brain signals.

12. The method of claim 11, further comprising receiving an additional brain signal and using the first decoder to determine whether the additional brain signal represents an intent to speak.

13. The method of claim 12, wherein the additional brain signal is from a patient, and wherein a frontal lobe of the patient is damaged such that the frontal lobe does not generate brain signals that result in speech.

14. The method of claim 12, further comprising, responsive to a determination that the additional brain signal represents the intent to speak, using the second decoder to identify one or more phonemes associated with the additional brain signal.

15. The method of claim 12, further comprising, responsive to a determination that the additional brain signal does not represent the intent to speak, taking no action to avoid reading thoughts.

16. The method of claim 12, wherein the brain signals comprise action potentials in the form of spikes.

17. The method of claim 12, wherein the brain signals comprise high gamma band signals.

18. The method of claim 12, wherein the brain signals are received from arrays of electrodes attached to brains of a plurality of individuals.

19. The method of claim 18, wherein the brain signals originate from one or both of the temporal lobe and the parietal lobe of the plurality of individuals.

20. The method of claim 12, wherein the binned features have a duration between 50 milliseconds and 100 milliseconds.
